SEQUENCE LISTING (1 sequence)

SEQ ID NO: 1 - DNA, 670 bp, Homo sapiens
ggctcctctccccaagtcaaaccttaccttacttaaccgattgtatttcctccctcagaggatgccatatctcagtacaggagagaaaatagaaggtagagagacttttacccaagcaccccctgaaccccaggtgtacacaactgaacctgatgcagtgacaatcacgttcacacataaaacatctggctaaaggctaagatcacttcggatttccgacatacattttcctaagcagtgcatttttctttaattttccttagaaaaagactgtaaagtagccccacaattcccacatcttcatactccaccctgcattcaagttttcctgggacaggtatctatgtgtgtgcatgaatctatttttacggcatatgtctaggaccccctacgaggagccaaagtttcagagagcccagcaactatgtaactccatggaagggaggcatgatattactctctgttcacaggagcgtacgcacagatcttttctcctcctcatggtcagttttctatttgtgattagtaattagcttctcttggtacgctacgatctattacaaaagccaaacattcagggggcgagctgaaatgacaaaatttggctataatttatgttggcccctgacatatatatttttttaatggtttggtctctaagcaactgatc

(19) United States
(12) Patent Application Publication
(10) Pub. No.: US 2009/0111105 A1
(43) Pub. Date: Apr. 30, 2009

(54) ANDROGEN-DEPENDENT 1-F-AROMATASE REPORTER GENE

(76) Inventors: Maik OBENDORF, Weimar (DE); Vladimir Patchev, Jena (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/167,463

(22) Filed: Jul. 3, 2008

Related U.S. Application Data
(60) Provisional application No. 60/947,711, filed on Jul. 3, 2007.

(30) Foreign Application Priority Data
Jul. 3, 2007 (EP) 07075554.1

(51) Int. Cl. C12Q 1/68; C12N 15/11

(52) U.S. Cl. 435/6; 536/24.1

(57) ABSTRACT

The present invention relates to an androgen-dependent 1-f-aromatase reporter gene and a method for the production of the 1-f-aromatase-reporter gene and use thereof in a method for identifying ligands of the androgen receptor.

This application claims the benefit of the filing date of U.S. Provisional Application Ser. No. 60/947,711 filed Jul. 3, 2007.

The present invention relates to an androgen-dependent 1-f-aromatase reporter gene and a method for the production of the 1-f-aromatase-reporter gene and use thereof in a method for identifying ligands of the androgen receptor.

Aromatase expression induced by androgens in the central nervous system and hence the formation of oestrogens is extraordinarily important in the establishment of sex-specific behavioural reactions, including the imprinting of sex identity, and the regulation of the libido, in particular in the male sex. In addition, aromatase expression induced by androgens in the brain appears to have an important role in processes such as learning and memory (Wickelgren, I.; Science, Vol. 276: 675-678, 1997). Positive effects from oestrogens have also been described in neuroregenerative processes (Abe-Dohmae, et al., J. Neurochem., Vol. 67: 2087-2095, 1996; I. Azcoitia et al., 2001, J. Neurobiol. 47: 318-329). Induction or upregulation of the oestrogen receptor and of neuronal oestrogen production seems to be part of a repair mechanism in various neuronal injuries (Dubal et al., 1999, J Neurosci 19:6385-6393; Garcia-Segura et al., 1999, Neuroscience 89: 567-578; Peterson et al., 2001, J Neuroendocrinol 13: 317-323; Carswell et al., 2005, J. Steroid Biochem. Mol. Biol. 96:89-91). Oestrogens therefore represent an important pharmacological option for the treatment or prevention of neurodegenerative diseases. However, their use, in both sexes, is restricted owing to possible side effects in other organs.

At present, no in vitro test systems are available for determining aromatase expression induced by androgens and thus the brain-specific synthesis of oestrogens and for identifying and characterizing substances that influence aromatase activity.

In vivo investigations with the end points oestrogen concentration in the brain, aromatase expression in the brain, aromatase activity in brain samples or behavioural testing in both sexes are known from the prior art.

As already mentioned, androgens induce aromatase expression, mediated by their binding to the androgen receptor (AR). The AR can either be activated or inactivated. Examples of activation in the case of androgen deficiency occur for example in sarcopenia, hypogonadism, age-related hypogonadism and forms of disturbance of libido and in erectile dysfunction in men.

Pharmacological inactivation of the AR is important e.g. in benign and malignant diseases of the prostate or in diseases that are linked to androgen excess, e.g. certain forms of acne, hair loss, or polycystic ovary and hirsutism in women.

As a rule, however, complete activation or deactivation of the androgen receptor is not desirable, but rather a tissue-selective action. For example, in the case of male hypogonadism, AR-mediated activation of the libido, of temperament and a bone- or muscle-anabolic action is desirable, but no activation, rather a neutral or an attenuating action on the prostate or the skin. In the case when deactivation of the AR is desirable, e.g. in malignant prostatic carcinomas or alopecia, deactivation of the AR is disadvantageous with respect to libido, bone metabolism etc. (S. S. Wolf and M. Obendorf, 2004, “Selective androgen receptor modulators (SARMs)”; in E. Nieschlag & H. M. Behre: ‘Testosterone’ 3rd. Edition; Cambridge University Press, ISBN 0521833809; 623-640).

Hitherto, cell culture or in vitro based assay systems have been inadequate for finding and characterizing these tissue-selective androgens or antiandrogens. This also applies to determination of aromatase expression.

Accordingly, the aim is to provide a method by which pharmacologically active substances can be identified and characterized, said substances being characterized in that they
bring about a selective increase in aromatase expression in the CNS and/or
have a tissue-selective influence on the activity of the AR.

This aim is achieved with a method in which, through stimulation of aromatase expression in a reporter gene assay, substances can be identified that selectively regulate oestrogen synthesis in the brain and exert a tissue-selective influence on the androgen receptor. It has been shown that there is androgen-dependent regulation of the 1f-aromatase promoter.

Human aromatase is encoded in a gene that is located on chromosome 15 (Chen, S. et al., (1988): Molec. Biol., Vol. 7: 27-38).

The human P450ₐᵣₒₘ gene (CYP19) comprises a 30 kb coding region and an approx. 93 kb regulatory region (Bulun, S. E. et al., Semin Reprod Med. Vol. 22: 5-9, 2004). The transcript consists of 10 exons, exon I being noncoding. The coding exons II to X are expressed identically in all tissues. To date, splicing variants in the coding regions have not yet been found in humans.

For exon I, several variants were discovered during sequencing of the mRNA from various tissues (for a review, see: Harada, N.; Utsumi, T. and Takagi, Y. Proc. Natl. Acad. Sci. USA, 90: 11312-11316, 1993, and Simpson, E. R.; Endocrine Reviews, 15: 341-355, 1994), which are expressed tissue-specifically and all can be linked to the same splicing site located at the start of exon II (FIG. 1). The tissue-specific occurrence of the exon-I sequences in mRNA can be explained by the use of alternative promoters, which are located in the gene before the respective exon-I variants. The activation of these various promoters is subject to control that has not yet been fully elucidated.

Table 1 lists examples of currently known uses of certain 5′-untranslated exons of human aromatase in various tissues according to Harada et al. (1993, Proc. Natl. Acad. Sci. USA, Vol. 90: 11312-11316).

Investigations to date, in particular of the two teams around Harada (Harada, N. et al., (1993) Proc. Natl. Acad. Sci. USA, Vol. 90: 11312-11316) and Simpson (Simpson, E. R.; Endocrine Reviews, Vol. 15: 341-355, 1994) showed, by sequencing the aromatase-mRNA, that in most tissues several promoters can be used and conversely one promoter can also be used in different tissues (Table 1).

An exception to this seems to be the promoter 1f, which as far as is known is used in humans exclusively in neuronal tissues.

In the brain, androgens in particular have an effect on aromatase expression (Abdelgadir, S. E. et al., Endocrinol., Vol. 135: 395-401, 1994; Rosselli, C. E. et al., (1996) Endocrine, Vol. 5: 59-65; Rosselli, C. E. et al., (1997, Brain Research, Vol. 44:351-357). Despite this finding, the precise molecular mechanism by which the androgen receptor (AR) regulates aromatase expression in the brain is still unknown. It is true that a putative AR responsible element in the 1f-aromatase promoter is mentioned in the literature, but a functional involvement of the 1f-promoter in the AR-mediated regulation of neuronal aromatase expression has not yet been shown (Honda, S. et al., Biochem Biophys Res Commun 198 (1994), 1153-1160. On the contrary: the team that first described the 1f-aromatase promoter (S. Honda, et al., Biochem Biophys Res Commun. 198 (1994), 1153-1160) points in later works to other regulators, which are not further characterized (Honda, S. I. et al., J Steroid Biochem Mol. Biol. 79:255-60, 2001; Honda, S. et al., Brain Res Mol Brain Res. 66: 122-32, 1999).

The basis of the present invention is the observation that control of the 1f-aromatase exon is regulated exclusively by the activities of the 1f-aromatase promoter and is not dependent on the cellular context. By means of an in vitro test system it was now possible to show the influence of pharmacologically active substances on the regulation of neuronal aromatase expression, without having to use neuronal tissue, neuronal cells, whole animals or extracts from the latter.

Use of such a test system makes possible and simplifies the discovery and characterization of pharmacologically active substances that either produce an increase in aromatase expression selectively in the central nervous system or are suitable for exerting a tissue-selective influence on the activity of the AR.

The object of the present invention is therefore an in vitro test system for the identification and characterization of substances that either bring about an increase in aromatase expression selectively in the central nervous system or are suitable for exerting a tissue-selective influence on the activity of the AR, said test system containing a fusion of reporter gene and aromatase promoter.

The tissue-specific regulation of aromatase, i.e. the key enzyme in oestrogen synthesis, is ultimately responsible for the targeted provision of oestrogens in the various tissues.

For characterization of hormone-controlled aromatase expression, established human cell lines are suitable for these in vitro tests, correlation with the tissue-specific in vivo expression being ensured by the use of various promoters. The development of a reporter gene assay is, in the case of the aromatase promoter, a simple and elegant possibility for testing the tissue-specific influence of particular substances on expression by activation of an individual promoter. In the case of the desired brain-specific expression, this is the 1f-aromatase promoter according to the invention.

By comparing the activity of the 1f-aromatase promoter (1f-aro-promoter), which is regulated in neuronal tissue by the AR, with the activity of a promoter that is regulated in another tissue by the AR or in general is regulated by AR, in vitro characterization of substances active on the AR with respect to tissue-selectivity is possible. The promoter activity of the 1f-aromatase promoter then serves as surrogate marker for AR-mediated activity in neuronal tissues or in an even more generalized manner as surrogate marker for AR-mediated activity other than in the gonadal tissue (e.g. in men, other than in the testis and prostate; in women, other than in the ovary).

By means of a reporter gene assay with the 1f promoter of human aromatase, it is possible to determine the regulation of neuronal aromatase expression. The neuronal 1f-aromatase promoter, whose sequence is known (Seq ID #1), is put in front of a reporter gene. The promoter-reporter gene fusion is transfected into a target line in a transient or stable manner. Expression of luciferase takes place as a function of the 1f-promoter, and the activity of the aromatase promoter can be determined from the amount of enzyme encoded by the reporter gene. In the present examples, the firefly and the Renilla luciferase genes were selected as examples of reporter genes.

The present invention relates to a promoter-reporter gene fusion consisting of 1f-aromatase promoter according to Seq ID #1 and reporter gene, and the reporter gene can be selected from the group comprising a firefly or Renilla luciferase gene, although this choice must not be limiting, and wherein the fusion product can be expressed in a cell line in a stable or transient manner.

If in addition the AR is expressed in the cells, the influence of the AR-active test substances on the activity of the aromatase promoter is exerted via the AR. The action of the test substances can then be determined on the basis of the altered luciferase enzyme activities. Accordingly, pharmacologically active substances can be sought or characterized without having to work with primary neuronal tissue or primary neuronal cells or with whole animals or with extracts from them.

The present invention relates to the use of a promoter-reporter gene fusion in an in vitro test system, wherein the promoter-reporter gene fusion consists of a 1f-aromatase promoter according to Seq ID #1 and reporter gene and wherein the influence of test substances is determined in a high-throughput reporter gene assay in cell culture and information can be obtained concerning the regulation of aromatase in neuronal tissues. This method is used for identifying substances that influence aromatase expression by binding to the androgen receptor.

The present invention also relates to the use of the promoter-reporter gene fusion, wherein sequences that have >90%, preferably >95% homology to the sequence given under Seq ID # 1, are used in a cell culture system for identifying substances that exert an influence on aromatase regulation in the brain by binding to the androgen receptor.

Furthermore, using the test model according to the invention it is also possible to characterize the properties of androgenic substances, including AR agonists, antagonists and SARMs, in which the activity of the 1f-aromatase promoter is compared with the activity of a promoter that is regulated in another tissue via the AR or in general is regulated by AR. The promoter activity of the 1f-aromatase promoter then serves as surrogate marker for AR-mediated activity in neuronal tissues or in an even more generalized manner as surrogate marker for AR-mediated activity other than in the gonadal tissue (e.g. in men other than in the testis and prostate, in women other than in the ovary).

For this, the test system according to the invention with the neuronal 1f-aromatase promoter is combined with another promoter regulated by the AR. The latter reflects either a general androgenic action (e.g. the MMTV—promoter) or an androgenic action in another androgenic target organ (e.g. promoters that are activated in the prostate by the androgen receptor). The simultaneous use of two different reporter genes, for example Renilla luciferase on the 1f-aromatase promoter and firefly luciferase on a second promoter, permits simultaneous measurement of both promoter activities. This provides, simply and elegantly, information on the tissue-selective activity of test substances with pharmacological action via the AR. Otherwise, both promoters can also be inserted successively before the same reporter gene, which once again represents a considerable simplification relative to the prior art, which as a rule is an animal test. The neuronal promoter activity can be abstracted in a more generalized manner as surrogate activity of a promoter other than in the gonads.

The present invention also relates to a method for determining the tissue-selective action of substances that bind to the androgen receptor, comprising the following steps:
a. Determination of the modulation of aromatase regulation using the promoter-reporter gene fusion in a reporter gene assay in cell culture systems for identifying both agonistic and antagonistic activities,
b. Determination of the modulation of another androgen-dependent promoter and
c. Comparison of step a and b, to provide a complete in vitro characterization of substances with respect to selective AR modulation on the 1f-aromatase promoter or in direct comparison with e.g. prostate-specific or generally acting AR-regulated promoters. This method can be used for identifying substances that exert a tissue-selective influence on the AR.

The present invention also relates to the use of the promoter-reporter gene fusion in a reporter gene assay, wherein sequences that have >90%, preferably >95% homology to the sequence stated under Seq ID # 1 are used in a reporter gene assay in cell culture systems for identifying substances that modulate the activity of the androgen receptor and provide tissue-selective modulation of the regulation of genes regulated by the androgen receptor.

“Antagonists” are to be understood as molecules that bind to their corresponding receptors and which inhibit the initiation of the signal transduction pathway or pathways coupled with the receptor by the naturally occurring ligand or ligands. Usually the antagonists compete with the naturally occurring ligands of the receptor for binding to the receptor. However, other modifications of the receptor by molecules that prevent the signal transduction pathways coupled with the receptor being activated by the naturally occurring ligand or ligands are also possible (e.g. non-competitive, steric modifications of the receptor).

Receptor antagonists typically bind selectively to their particular receptor and not to other receptors. Normally they display greater binding affinity than the natural ligand. Although antagonists that have greater affinity for the receptor than the natural ligand are preferred, antagonists with lower affinity can also be used. Preferably the antagonists bind reversibly to their corresponding receptors.

“Agonists” are to be understood as molecules that bind to their corresponding receptors and usually compete with the naturally occurring ligands of the receptor for binding to the receptor and which stimulate the initiation of the signal transduction pathway coupled to the receptor. Agonists can also support the binding of the natural ligands.

Receptor agonists typically bind selectively to their particular receptor and not to other receptors. They normally have greater binding affinity than the natural ligand. Although agonists that have greater affinity for the receptor than the natural ligand are preferred, agonists with lower affinity can also be used. Preferably the agonists bind reversibly to their corresponding receptors. Agonists are tested by signal transduction mediated by initiation of the corresponding receptor and/or physiological action.

“Ligands” denote the compounds or low-molecular substances that bind to a receptor. Their binding is usually reversible. The binding of a ligand to the corresponding receptor activates or inactivates the signal transduction pathway coupled to the receptor. This is how the ligand imparts its intracellular action.

Ligands are to be understood as agonists and antagonists of a receptor.

### BIOLOGICAL EXAMPLES

### 1. Cloning of the Human 1f-Aromatase Promoter in Reporter Gene Plasmids

For the reporter gene assay, the 1f-promoter of human aromatase is amplified from genomic DNA from human SH-SY5Y cells. Isolation of genomic DNA is carried out using the QIAamp Blood Kit according to the protocol specified by the manufacturer (Qiagen). The 1f-promoter of aromatase is known from publications (S. Honda, et al., Biochem Biophys Res Commun 198 (1994), pp. 1153-1160) and from the gene bank (CYP19A1). Using specific primer pairs (Table 2), portions of the human aromatase-promoter region including the 1f-promoter are amplified by PCR.

For amplification of DNA-fragments by PCR, in each case 1 μl of the genomic DNA samples is used. This is denatured for 2 min at 94° C. and amplified with the stated DNA primers EbrainB4 and Ebrain-c670 in 35 cycles, analysed by gel electrophoresis (FIG. 2) and purified of by-products by DNA extraction from agarose gel.

The PCR product of the 1f-promoter fragment possesses 3′-A overhangs and is ligated with the linear pTAdv cloning vector (FIG. 3). The pTAdv vector was obtained from Clontech. The insert of the resultant 1f-pTAdv2 vector (FIG. 4) is sequenced and the amplified human 1f-aromatase promoter sequence confirmed (Seq ID #1). It differs from the sequence published previously (S. Honda et al., N Biochem Biophys Res Commun 198 (1994), 1153-1160), but tallies with more recent gene bank entries.

The reporter gene used is for example the luciferase gene of the pGI3 vector from Promega. For production of the promoter-reporter gene construct with the 1f-promoter, the control promoter (SV40) with length of 213 bp is cut out of the vector pGI3-control (FIG. 5) with HindIII and XhoI. The pGI3 vector thus linearized and shortened is purified by gel electrophoresis and DNA extraction, dephosphorylated, then the fragment of the 1f-promoter that was cut out with HindIII and XhoI from the 1f-pTAdv2 vector (FIG. 4) and purified, is spliced in and the reporter gene plasmid 1f-pGI3 is produced (FIG. 6).

### 2. Transfection Experiments for Determining the Influence of Hormonally Active Compounds on the 1f-Promoter in Reporter Gene Assays in Cell Culture

PC3 (human prostate carcinoma) cells (from DSMZ GmbH, Braunschweig) are passaged in RPMI 1640 medium with phenol red (Order No.: 31870-025 from Gibco)+10% fetal calf serum (FCS) with 200 mM glutamine and 5 mg/l penicillin/streptomycin and cultivated at 37° C. with 5% CO₂ and 100% relative humidity in incubators (Heraeus). Before the tests the PC3-cells are passaged twice with test medium; this contains no phenol red (Order No.: 32404-014 from Gibco) and fetal calf serum, which was purified beforehand with activated carbon to remove hormone residues (DCS).

In order to determine the influence of hormones on the cloned 1f-aromatase promoter in the pGI3 fusion vector 1f-pGI3 on the basis of the luciferase activity, in 6-well plates 200000 cells per well are cotransfected with 0.2 μg of this plasmid with 0.5 μg of the pSG5AR plasmid. The pSG5AR vector is based on the pSG5 vector, which possesses the SV40 early gene promoter, which in human cells leads to constitutive expression of the underlying genes (Breathnach, R. and Harris, B. A. 1983, Nucleic Acids Res. 11:7119-36), in this case of the gene for the AR. Transient transfection with the reporter gene plasmid and the pSG5AR plasmid is carried out with lipofectin according to the instructions of GibcoBRL [Lipofectin-Reagent package insert, Cat.No.: 18292-037]. 20 h after transfection, the androgen dihydrotestosterone (DHT) is added. After incubation for a further 24 hours in the incubator, the cells are washed with PBS, lysed and the reporter gene activity is determined. Luciferase activity is determined using luciferase assay substrate according to the instructions of Promega [Luciferase Assay Substrate package insert, Cat.No.: E151A from: Luciferase Assay System with Reporter Lysis Buffer; Part TB161, USA 3/98]. To standardize the measured values, which are stated in light units, they can be divided by the amount of protein used in μg, stating the result in relative light units (RLU).

The 1f-reporter gene product shows, in the transfection carried out in the prostate cell line PC3, induction of reporter gene expression with DHT by a factor of 2.6 (FIG. 7). The regulation by androgens can be determined here in vitro.

### 3. Androgen-Dependent Regulation of the 1f-Aromatase Promoter

In addition to the prostate PC3 cells, a neuronal cell line is used for analysis of androgen-regulated reporter gene expression on the 1f-aromatase promoter.

The human neuroblastoma cell line SH-SY5Y (from DSMZ GmbH, Braunschweig) is passaged in Dulbecco's MOD Eagle Medium with phenol red (Order No.: 31885-023 from Gibco)+15% fetal calf serum (FCS) with 5 mg/l penicillin/streptomycin and cultivated at 37° C. with 5% CO₂ and 100% relative humidity in incubators (Heraeus). Before the tests, the SH-SY5Y cells are passaged twice with test medium; this contains no phenol red (Order No.: 11880-028 from Gibco) and fetal calf serum, which was purified beforehand with activated carbon to remove hormone residues (DCS).

In order to determine the influence of hormones on the cloned 1f-aromatase promoter in the pGI3 fusion vector 1f-pGI3 on the basis of the luciferase activity, in 6-well plates 300000 cells per well are cotransfected with 0.2 μg of this plasmid with 0.5 μg of the pSG5AR plasmid. The pSG5AR vector is based on the pSG5 vector, which possesses the SV40 early gene promoter, which in human cells leads to constitutive expression of the underlying genes (Breathnach, R. & Harris, B. A. 1983; “Plasmids for the cloning and expression of full-length double-stranded cDNAs under control of the SV40 early or late gene promoter”, Nucleic Acids Res. 11:7119-36), in this case of the gene for the AR. Transient transfection with the reporter gene plasmid and the pSG5AR plasmid is carried out with lipofectin according to the instructions of GibcoBRL [Lipofectin-Reagent package insert, Cat.No.: 18292-037]. 20 h after transfection, the androgen dihydrotestosterone (DHT) is added. After incubation for a further 24 hours in the incubator, the cells are washed with PBS, lysed and the reporter gene activity is determined. Luciferase activity is determined using luciferase assay substrate according to the instructions of Promega [Luciferase Assay Substrate package insert, Cat.No.: E151A from: Luciferase Assay System with Reporter Lysis Buffer; Part TB161, USA 3/98]. To standardize the measured values, which were stated in light units, they can be divided by the amount of protein used in μg, stating the result in relative light units (RLU).

The 1f-reporter gene product shows, in the transfection carried out in the neuroblastoma cell line SH-SY5Y, concentration-dependent induction of reporter gene expression with DHT (FIG. 8).

As similar effects are measured in both cell lines used, the activity of the 1f-promoter in these test systems is stimulated by androgens independently of the cellular context.

### 4. Transfection Experiments for Characterization of Hormonal Regulation of the 1f-Aromatase Promoter by Pharmacologically Active Substances

As well as DHT, on the basis of the 1f-luciferase reporter gene construct (1f-pGI3) as further pharmacologically active substance we tested the influence of estradiol on the expression of human aromatase in neuronal tissues (FIG. 9). PC3 (human prostate carcinoma) cells (from DSMZ GmbH, Braunschweig) were passaged in RPMI 1640 medium with phenol red (Order No.: 31870-025 from Gibco)+10% fetal calf serum (FCS) with 200 mM glutamine and 5 mg/l penicillin/streptomycin and cultivated at 37° C. with 5% CO₂ and 100% relative humidity in incubators (Heraeus). Before the tests, the PC3-cells are passaged twice with test medium; this contains no phenol red (Order No.: 32404x-014 from Gibco) and fetal calf serum, which was purified beforehand with activated carbon to remove hormone residues (DCS).

In order to determine the influence of estradiol (E2) on the cloned 1f-aromatase promoter in the pGI3 fusion vector 1f-pGI3 on the basis of the luciferase activity, in 6-well plates 200000 cells per well are cotransfected with 0.2 μg of this plasmid with 0.5 μg of the pSG5-ER plasmid. The pSG5-ER vector is based on the pSG5 vector, which possesses the SV40 early gene promoter, which in human cells leads to constitutive expression of the underlying genes (Breathnach, R. & Harris, B. A. 1983; “Plasmids for the cloning and expression of full-length double-stranded cDNAs under control of the SV40 early or late gene promoter”, Nucleic Acids Res. 11:7119-36), in this case of the gene for human oestrogen receptor alpha (ERα). Transient transfection with the reporter gene plasmid and the pSG5-ER plasmid is carried out with lipofectin according to the instructions of GibcoBRL [Lipofectin-Reagent package insert, Cat.No.: 18292-037]. 20 h after transfection, the androgen dihydrotestosterone (DHT) is added. After incubation for a further 24 hours in the incubator, the cells are washed with PBS, lysed and the reporter gene activity is determined. Luciferase activity is determined using luciferase assay substrate according to the instructions of Promega [Luciferase Assay Substrate package insert, Cat.No.: E151A from: Luciferase Assay System with Reporter Lysis Buffer; Part TB161, USA 3/98]. To standardize the measured values, which are stated in light units, they can be divided by the amount of protein used in μg, stating the result in relative light units (RLU).

Direct influencing of neuronal aromatase expression or activity by means of oestrogens is not described in the literature. Also in the 1f-reporter gene assay according to the invention, no effect of estradiol on 1f-promoter activity can be measured (FIG. 9). This finding also coincides with a sequence analysis of the 1f-promoter, in which at least no known ERE sequences are contained. An influence of the oestrogen concentration on aromatase activity in the brain is admittedly discussed in the literature (Rosselli, C. E. and Resko, J. A. (1993): Aromatase activity in the rat brain: hormonal regulation and sex differences; J. Steroid. Biochem., Vol. 44: 499-508), although to the effect that this influence is mediated by a feedback mechanism. The oestrogens produced by the aromatase lower the expression of the androgen receptors and hence indirectly also the expression of aromatase, as the presence of the androgen receptor is essential for the androgenic action to take effect. This feedback regulation is not present in the in vitro reporter gene assay according to the invention, as the receptor gene plasmids based on the pSG5 vector lead to expression of the receptors independently of the oestrogen concentration. This switching-off of the feedback mechanisms present in the complex in vivo system permits, in the transactivation assays that were carried out, investigation of the direct regulation of aromatase expression at promoter level. The reporter gene assay according to the invention is therefore the most suitable for further more detailed elucidation of the necessary stimuli for direct regulation of neuronal aromatase expression.

### 5. Transfection Experiments for Identification and Characterization of Pharmacologically Active Substances that Regulate Neuronal Aromatase Expression

As well as DHT, other pharmacologically active androgens are tested with the 1f-luciferase-reporter gene construct (FIG. 10).

The human neuroblastoma cell line SH-SY5Y (from DSMZ GmbH, Braunschweig) was passaged in Dulbecco's MOD Eagle Medium with phenol red (Order No.: 31885-023 from Gibco)+15% fetal calf serum (FCS) with 5 mg/l penicillin/streptomycin and cultivated at 37° C. with 5% CO₂ and 100% relative humidity in incubators (Heraeus). Before the tests, the SH-SY5Y cells are passaged twice with test medium; this contains no phenol red (Order No.: 11880-028 from Gibco) and fetal calf serum, which was purified beforehand with activated carbon to remove hormone residues (DCS).

In order to determine the influence of hormones on the cloned 1f-aromatase promoter in the pGI3 fusion vector 1f-pGI3 on the basis of the luciferase activity, in 6-well plates 300000 cells per well are cotransfected with 0.4 μg of this plasmid with 0.5 μg of the pSG5AR plasmid. The pSG5AR vector is based on the pSG5 vector, which possesses the SV40 early gene promoter, which in human cells leads to constitutive expression of the underlying genes (Breathnach, R. & Harris, B. A. 1983; “Plasmids for the cloning and expression of full-length double-stranded cDNAs under control of the SV40 early or late gene promoter”, Nucleic Acids Res. 11:7119-36), in this case the gene for the AR. Transient transfection with the reporter gene plasmid and the pSG5AR plasmid is carried out with lipofectin according to the instructions of GibcoBRL [Lipofectin-Reagent package insert, Cat.No.: 18292-037]. 20 h after transfection, the test substances are added. After incubation for a further 24 hours in the incubator, the cells are washed with PBS, lysed and the reporter gene activity is determined. Luciferase activity is determined using luciferase assay substrate according to the instructions of Promega [Luciferase Assay Substrate package insert, Cat.No.: E151A from: Luciferase Assay System with Reporter Lysis Buffer; Part TB161, USA 3/98]. To standardize the measured values, which are stated in light units, they can be divided by the amount of protein used in μg, stating the result in relative light units (RLU).

In the transfection that was carried out, the 1f-reporter gene plasmid shows a concentration-dependent induction of reporter gene expression with DHT (FIG. 8). As well as DHT, other androgens are also tested with the 1f-luciferase-reporter gene construct (FIG. 10). All androgens stimulate the 1f-promoter, but with slightly different potency in each case. Activation can also be observed with MENT and testosterone propionate. The very potent stimulation by testosterone propionate in the relative comparison tallies with the good clinical effects of testosterone derivatives on male libido (Gooren, 1987, Arch. Sex. Beh. 16:463-473; Bancroft 1988, In Handbook of Sexology 6, 297-315 Elsevier, Amsterdam, Buna et al. 1993, Fertil. Steril. 59: 1118-1123), which in primates is controlled primarily by activation of neuronal aromatase expression (Zumpe et al., 1993, Horm. Behav. 27:200-215).

### 6. Transfection Experiments for Identification and Characterization of Tissue-Selective Androgens, which Regulate AR-Regulated Genes in Neuronal Tissue Differently Compared with Other Tissues

The test system according to the invention with the neuronal 1f-aromatase promoter is combined with the MMTV-promoter, which reflects a general androgenic action (Parker, M. G. et al., J Cell Biochem. 1987, 35:285-92). The hormone-regulated MMTV-promoter is cloned in front of the firefly luciferase gene of the pGL3control vector from Promega, to produce the vector MMTV-luc.

The human neuroblastoma cell line SH-SY5Y (from DSMZ GmbH, Braunschweig) is passaged in Dulbecco's MOD Eagle Medium with phenol red (Order No.: 31885-023 from Gibco)+15% fetal calf serum (FCS) with 5 mg/l penicillin/streptomycin and cultivated at 37° C. with 5% CO₂ and 100% relative humidity in incubators (Heraeus). Before the tests, the SH-SY5Y cells are passaged twice with test medium; this contains no phenol red (Order No.: 11880-028 from Gibco) and fetal calf serum, which was purified beforehand with activated carbon to remove hormone residues (DCS).

In order to determine the influence of hormones on the cloned 1f-aromatase promoter in the pGI3 fusion vector on the basis of the luciferase activity in comparison with the MMTV-promoter, in parallel transfection experiments 1.5 μg of the 1f-pGI3 plasmid or of the MMTV-luc plasmid is transfected together with 0.75 μg of the pSG5AR plasmid in 300000 SH-SY5Y cells per well of a 6-well plate. Transient transfection with the reporter gene plasmids is carried out with lipofectin according to the instructions of GibcoBRL [Lipofectin-Reagent package insert, Cat.No.: 18292-037]. 20 h after transfection, the test substances are added. After incubation for a further 24 hours in the incubator, the cells are washed with PBS, lysed and the reporter gene activity is determined. Luciferase activity is determined using luciferase assay substrate according to the instructions of Promega [Luciferase Assay Substrate package insert, Cat.No.: E151A from: Luciferase Assay System with Reporter Lysis Buffer; Part TB161, USA 3/98]. To standardize the measured values, which were stated in light units, they are divided by the amount of protein used in μg and the relative light units per μg of protein used are stated as a percentage of the maximum stimulation.

In the transfections carried out in the neuroblastoma cell line SH-SY5Y, the 1f-reporter gene plasmid shows induction of reporter gene expression with the androgens tested, in this example DHT and oxymetholone. The MMTV-reporter gene plasmid also shows induction of reporter gene expression in both cases (FIG. 12). As expected, oxymetholone is weaker than DHT. Surprisingly and unpredictably, the substances differ, sometimes considerably, with respect to induction on the two promoter constructs (FIG. 11). Whereas oxymetholone produces very similar activation on both promoters tested, DHT shows marked differences between the two promoters and is less potent on the neuronal 1f-aromatase promoter compared with the MMTV promoter. These differences point to a possible tissue-selective difference in vivo and therefore in humans. It is known from animal tests that DHT, in comparison with testosterone for example, has considerably less action on male sexual behaviour, whereas it acts as a very strong androgen on the prostate (Arteaga-Silva, M. et al., 2005, Physiol Behav. 85: 571-80). This behaviour of DHT was also confirmed in comparison with oxandrolone in an extensive in vivo experiment, which took 4 months (FIG. 12). The in vitro test system according to the invention therefore permits, simply and elegantly, rapid identification and characterization of androgens with a possible tissue-selective action. It is therefore an ideal test system, with potential for high-throughput testing.

### 7. Transfection Experiment for Simultaneous Identification and Characterization, Also with Respect to Partial-Agonistic and Partial-Antagonistic Effects of Tissue-Selective Androgens Providing Tissue-Selective Differential Regulation of AR-Regulated Genes

The test system according to the invention with the neuronal 1f-aromatase promoter is combined with the MMTV-promoter, which reflects a general androgenic action (Parker, M. G. et al., J Cell Biochem. 1987, 35:285-92). Both androgen-regulated promoter activities can be measured simultaneously in a transfection assay or optionally in a stably transfected cell line, using two different reporter gene constructs. In the present example the hormone-regulated MMTV-promoter is cloned in front of the firefly luciferase gene of the pGL3control vector from Promega, to produce the vector MMTV-luc, whereas the 1f-aromatase promoter is cloned in front of the Renilla luciferase gene of the pRL vector from Promega, to produce the vector 1f-aromatase-pRL.

For optional simplification of the experimental setup, the AR is first transfected stably in a cell line. The cDNA of the AR is cloned into the expression plasmid pSG5, to generate the plasmid pSG5AR. This vector possesses the SV40 early gene promoter, which in human cells leads to constitutive expression of the underlying genes (Breathnach, R. and Harris, B. A. 1983, Nucleic Acids Res. 11:7119-36), in this case the gene of the AR. PC3 (human prostate carcinoma) cells (from DSMZ GmbH, Braunschweig) are stably transfected with the pSG5AR vector, with the additional use of a plasmid that encodes a resistance gene for the antibiotic G418. The resultant PC3-AR+ cells stably express human AR. PC3AR+ cells are passaged in RPMI 1640 medium with phenol red (Order No.: 31870-025 from Gibco)+10% fetal calf serum (FCS) with 200 mM glutamine, 5 mg/l penicillin/streptomycin and 200 g/ml Geniticin and cultivated at 37° C. with 5% CO₂ and 100% relative humidity in incubators (Heraeus). Before the tests the PC3-AR+were passaged twice with test medium; this contains no Geniticin, no phenol red (Order No.: 32404-014 from Gibco) and fetal calf serum, which is purified on activated carbon to remove hormone residues (DCS).

In order to determine the influence of hormones on the cloned 1f-aromatase promoter in the pRL fusion vector on the basis of Renilla activity in comparison with firefly luciferase activity of the MMTV-promoter, in transfection experiments 8 μg of the 1f-aromatase-pRL plasmid and 20 μg of the MMTV-luc plasmid are transfected in 300000 PC3-AR+ cells per 175 ml bottle. Transient transfection with the reporter gene plasmids is carried out with lipofectin according to the instructions of GibcoBRL [Lipofectin-Reagent package insert, Cat.No.: 18292-037]. 6 h after transfection, the cells are washed and distributed on 96-well microtitre plates, with 10000 cells per well. 16 h after transfer to 96-well plates, a selective androgen receptor modulator (SARM) is added, both on its own for measurement of agonistic activities and in the presence of 10×e⁻¹⁰ M testosterone for measurement of antagonistic activities. After incubation in the incubator for a further 20 hours, the cells are washed with PBS, lysed and the reporter gene activities are determined. The firefly and Renilla luciferase activities are determined in relative light units with a dual Luciferase Assay Kit (Order No.: E 1960 from Promega) in accordance with the manufacturer's instructions [Promega; Luciferase Assay Substrate package insert, Cat.No.: E151A from: Luciferase Assay System with Reporter Lysis Buffer; Part TB161, USA 3/98]. For optional standardization of the measured values, which were stated in light units, they can be divided by the amount of protein used in μg, stating the result in relative light units (RLU).

In the test for agonism, the SARM tested here, like the DHT already presented in the previous example, acts very differently on the two AR-dependent promoters (FIG. 13). In contrast to DHT, however, in this case there is no activation of the general MMTV promoter, whereas the neuronal 1f-aromatase promoter, which serves as an example of promoter of an AR-regulated promoter other than in the gonads, is activated. Like the previous example with DHT, this points to a possible tissue-selectivity also in vivo and in humans. Furthermore, the parallel measurements of antagonism can provide indications of possible selective modulation of AR activity. That is, beyond the state of the art, it is possible to obtain indications of SARM activity with respect to possible tissue-selective action.

As has also been clearly demonstrated in this example, the measurements can take place in the microtitre format, which underlines the basic high-throughput capability of the test system according to the invention. SARMs, which as in this example generally have clearly antagonistic action, but in specific tissues also display a certain agonistic action, are of particular interest for a great variety of indications (S. S. Wolf and M. Obendorf, 2004, in E. Nieschlag & H. M. Behre: ‘Testosterone’ 3rd Edition; Cambridge University Press, ISBN 0521833809; 623-640).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding European application No. 07 075 554.1, filed Jul. 3, 2007, and U.S. Provisional Application Ser. No. 60/947,711, filed Jul. 3, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

### FIGURES

FIG. 1:

Genomic promoter region of the human aromatase gene with the known splicing variants of the alternative exon-I variants (simplified representation). For greater clarity, portions of the 5′-region have been cut out and shown as omissions (modified from Harada, N. et al., Proc. Natl. Acad. Sci. USA, Vol. 90: 11312-11316, 1993 and Simpson, E. R. Endocrine Reviews, Vol. 15: 341-355, 1994).

FIG. 2:

PCR product with the 1f-aro-promoter, amplified from genomic DNA from SH-SY5Y cells. a: length standard b: PCR amplificate of 1f-aromatase promoter.

FIG. 3:

Map of the pTAdv vector. This linearized vector, provided with a 3′-T overhang at both ends, was used for the ligations with PCR fragments.

FIG. 4:

Vector map of the 1f-ptadv” vector with the amplified 1f-aromatase promoter-DNA in the pTAdv vector. HindIII and XhoI mark cleavage sites that were used for further cloning.

FIG. 5:

Vector map of the pGI3-control vector. HindIII and XhoI mark cleavage sites that were used for further cloning.

FIG. 6:

Vector map of the 1f-pGI3 vector. HindIII and XhoI mark cleavage sites that were used for production of the vector.

FIG. 7:

Dependence of activation of the 1f-promoter on the concentration of dihydrotestosterone (DHT) in PC3-cells. 0.2 μg of the 1f-pGI3 plasmid and 0.5 μg of the pSG5-AR plasmid are transfected. Incubation with DHT for 20 h. The mean values of double determinations are shown in relative light units (RLU) per μg total protein.

FIG. 8:

Dependence of activation of the 1f-promoter on the concentration of dihydrotestosterone (DHT) in SH-SY5Y cells. 0.2 μg of the 1f-pGI3 plasmid and 0.5 μg of the pSG5-AR plasmid are transfected. Incubation with DHT for 20 h. The mean values of double determinations are shown in relative light units (RLU) per μg total protein.

FIG. 9:

This shows that activation of the 1f-promoter is independent of the concentration of estradiol (E2) in PC3-cells. 0.2 μg of the 1f-pGI3 plasmid and 0.5 μg of the pSG5-ER plasmid are transfected. Incubation with E2 for 20 h. The mean values of double determinations are shown in relative light units (RLU) per μg total protein.

FIG. 10:

Influence of various androgens on the 1f-promoter in neuronal SH-SY5Y cells. 0.4 μg of the 1f-pGI3 and 0.5 μg of the pSG5-AR plasmid are transfected. Incubation with a) T (testosterone propionate), b) MENT (7α-methyl-19-nortestosterone) and c) DHT (dihydrotestosterone) for 20 h. The mean values from double determinations are shown in relative light units (RLU) per μg total protein.

FIG. 11:

Influence of various androgens on the 1f-promoter and the MMTV-promoter in neuronal SH-SY5Y cells. 1.5 μg of the 1f-pGI3 and of the MMTV-luc promoter and 0.75 μg of the pSG5-AR plasmid are transfected. Incubation with oxymetholone or DHT (dihydrotestosterone) for 20 h. The standardized mean values of the relative light units (RLU) are shown as percentage from quadruple determinations with standard deviations.

FIG. 12:

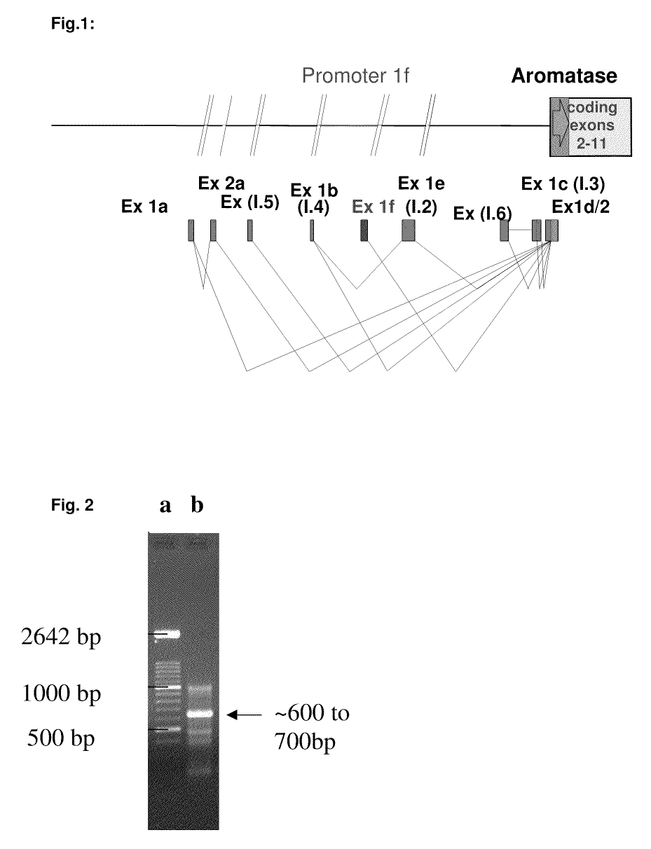

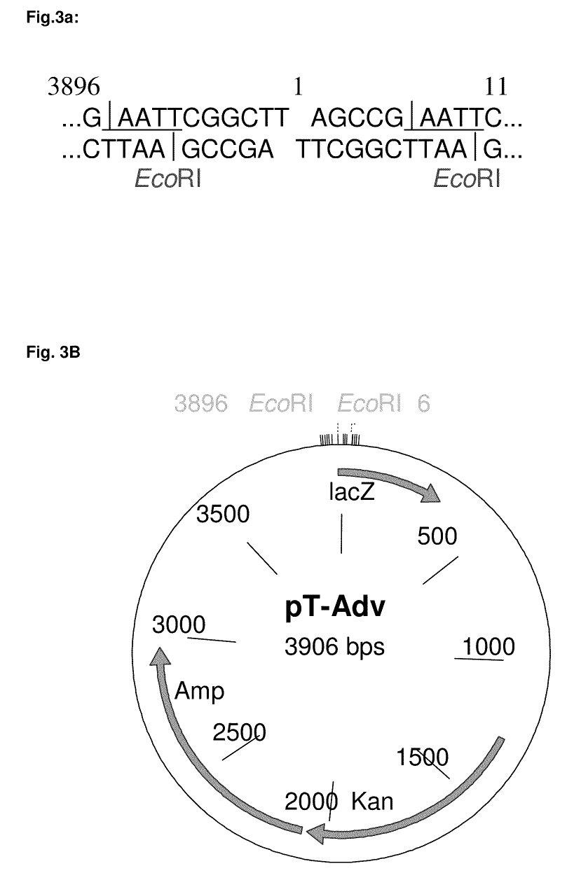

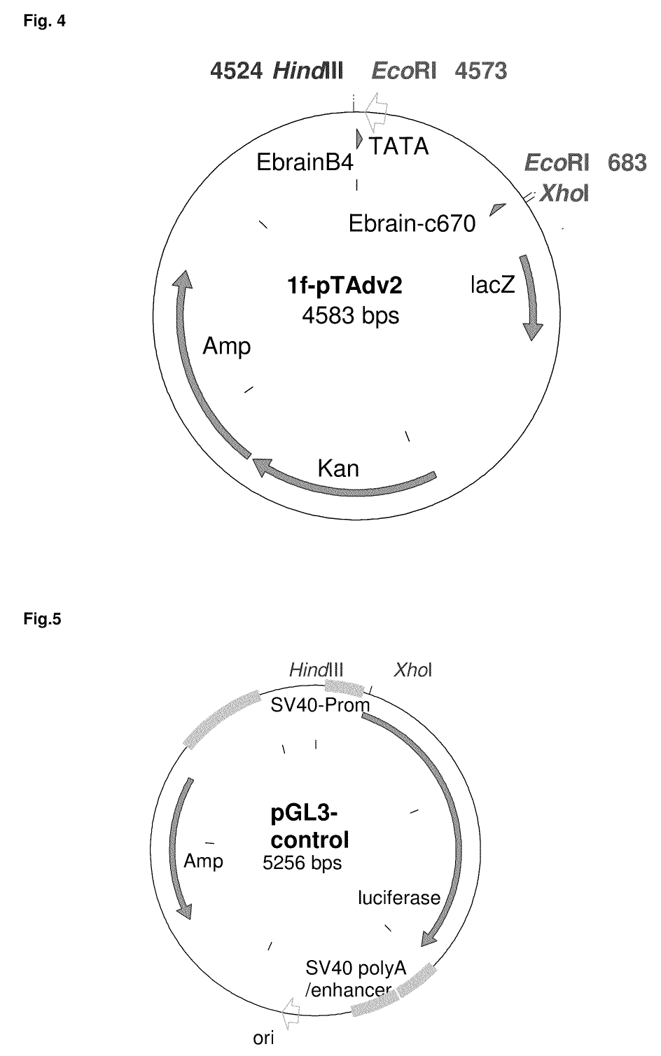

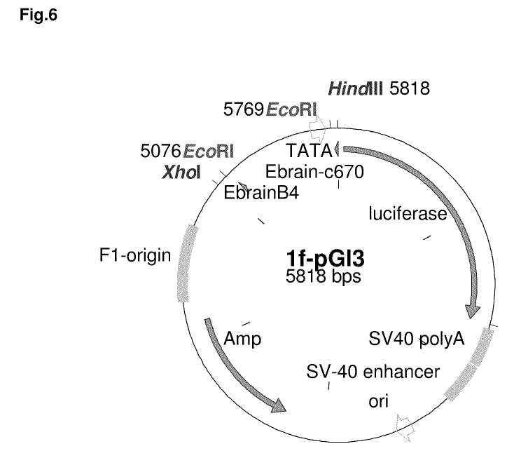

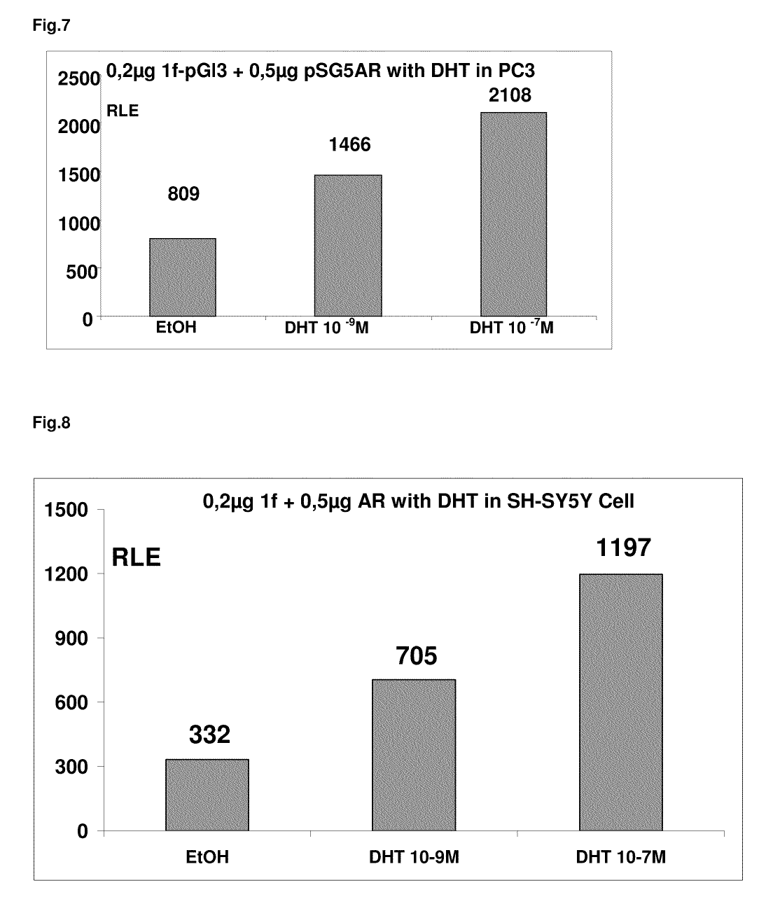

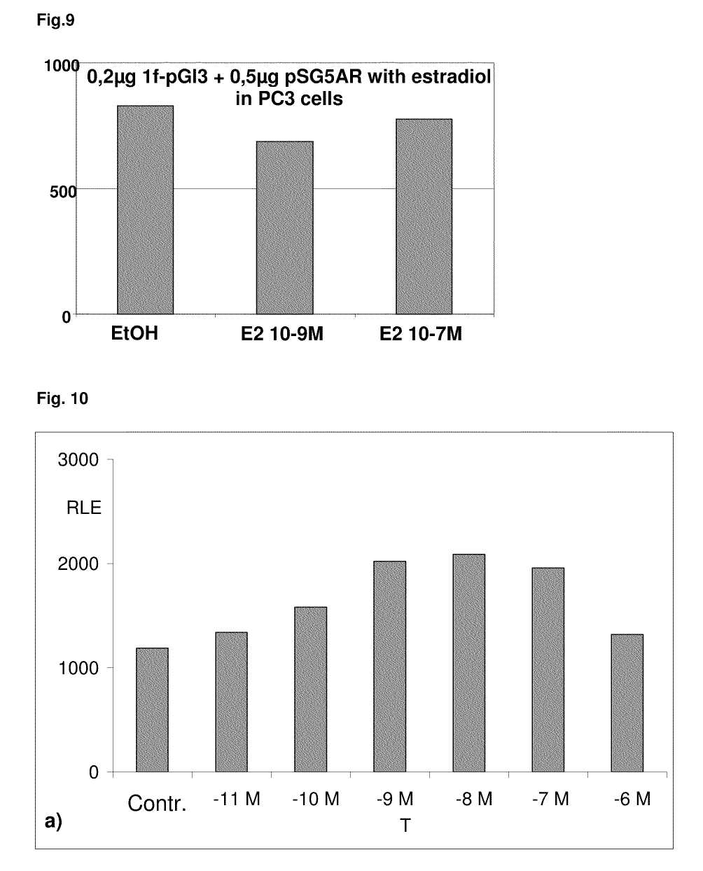

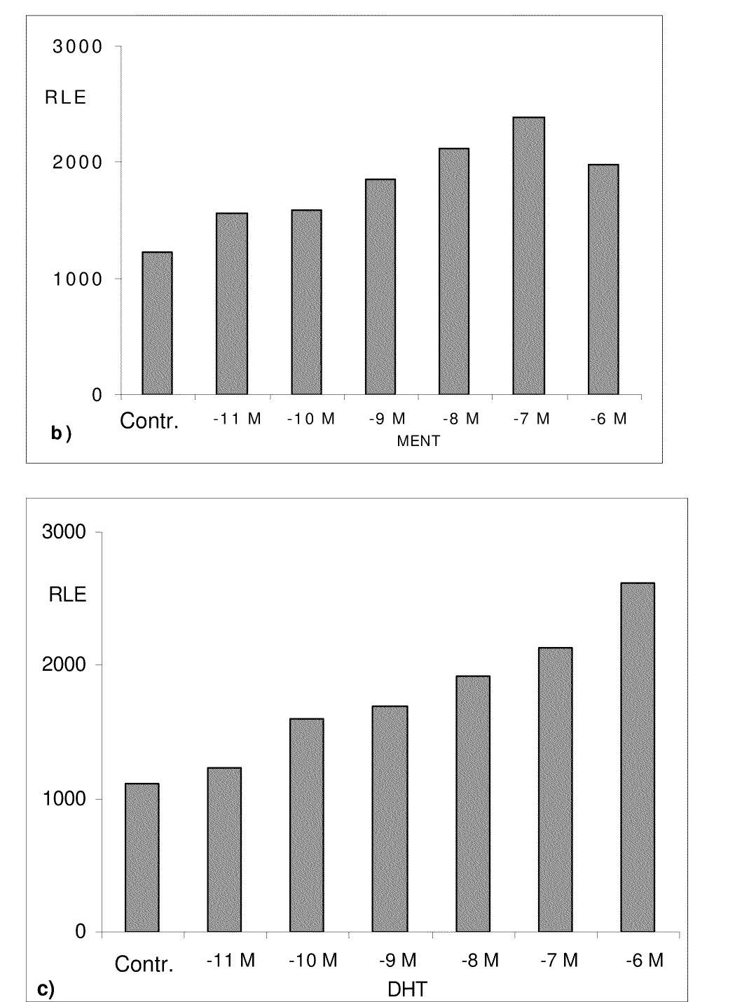

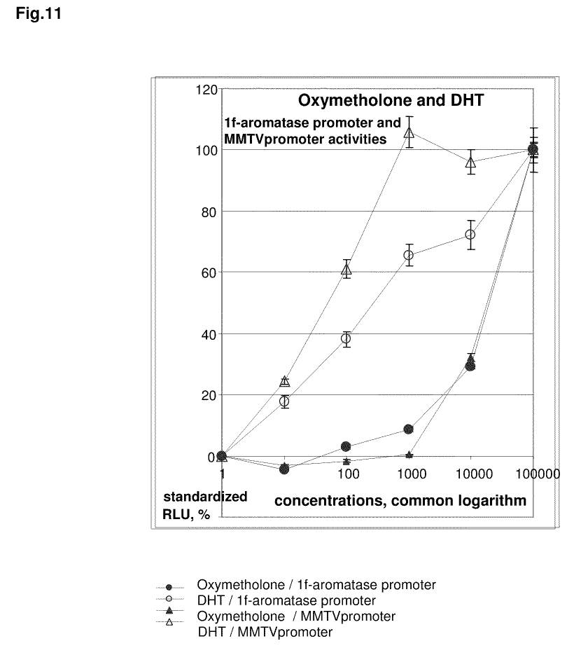

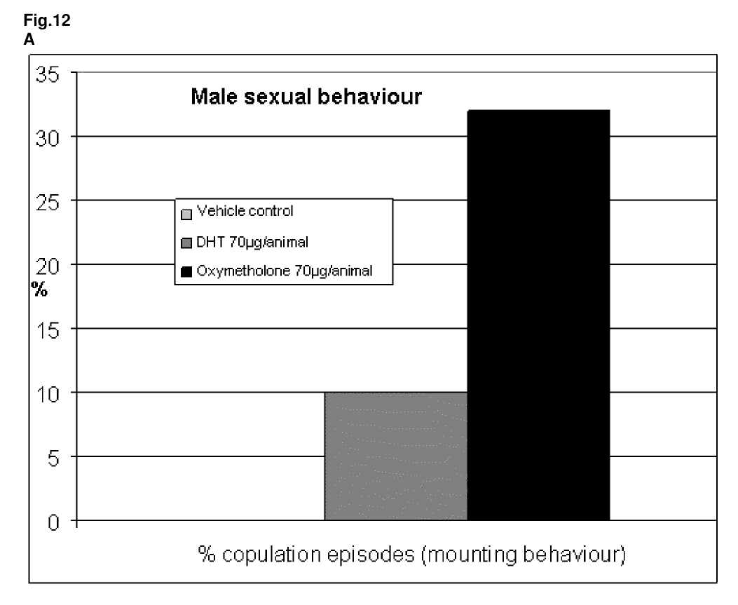

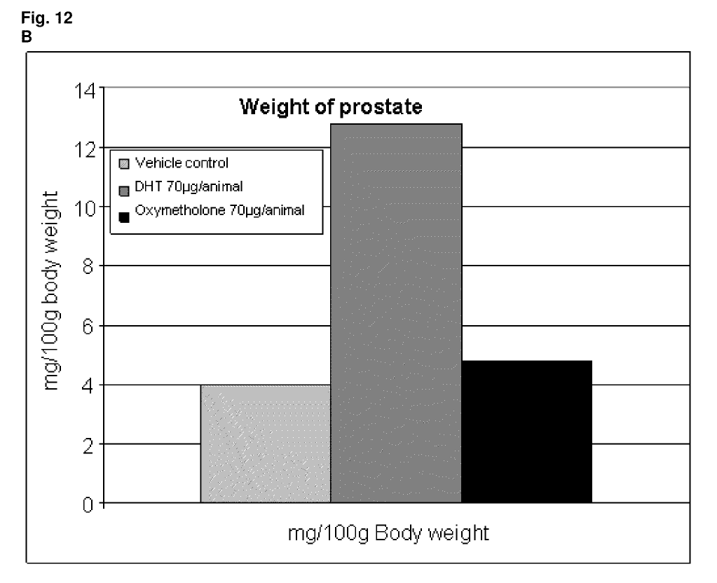

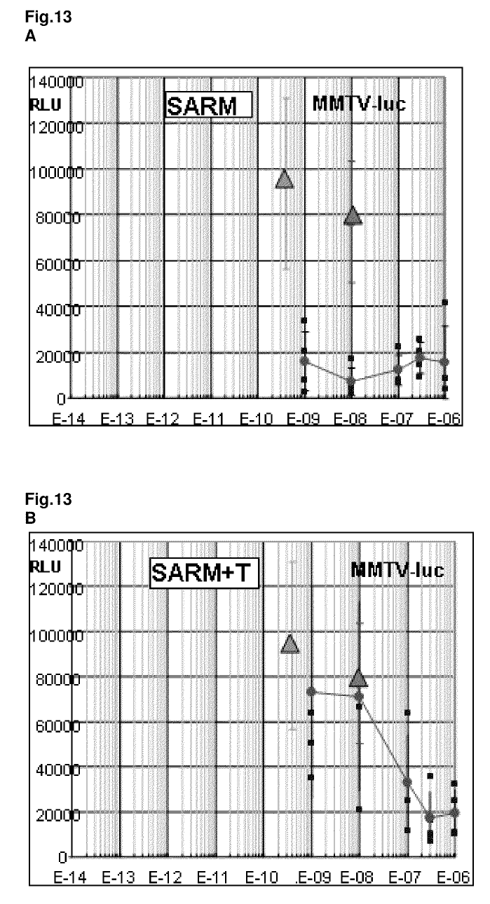

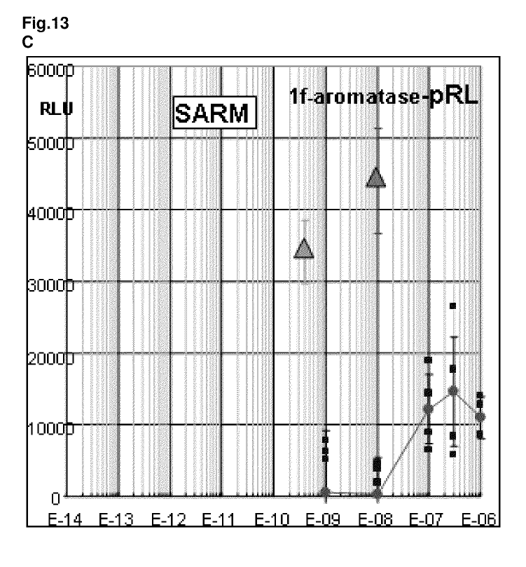

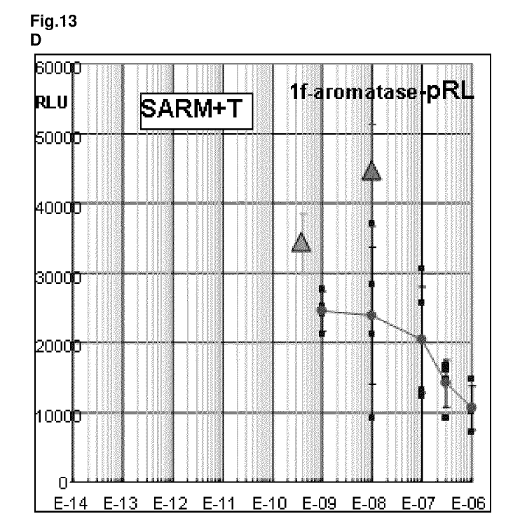

In vivo influence of various androgens on male sexual behaviour (A), which represents a neuronal end point of the androgenic action, and on the weight of the prostate (B), which represents a gonadal end point of the androgenic action. Intact male rats with sexual experience are selected and castrated. After 7 weeks and loss of sexual activity, the animals are divided into three groups with 9-10 animals per group and are treated for one week with 1050 μg oxymetholone, DHT or vehicle. From day 8 to day 120, the animals receive daily 70 μg oxymetholone (black), DHT (dark grey) or vehicle (light grey). Each animal was tested 7 times for sexual behaviour (mounting behaviour). 4 months after the start of the study, the organs were removed and prostate and body weights were determined.

FIG. 13:

Testing for agonism (A+C) and antagonism (B+D) of a selective AR modulator (SARM) simultaneously on the nonspecific MMTV-promoter (A+B) and on the neuronal 1f-aromatase promoter (C+D) in pC3AR+ cells. 1.5 μg of the 1f-pGI3 and of the MMTV-luc promoter and 0.75 μg of the pSG5-AR plasmid are transfected.

Incubation with the SARM for 20 h. In the tests for antagonism (B+D) the SARM is incubated at the stated concentration in the presence of 4×10 e⁻¹⁰ M testosterone. The individual values are shown as squares and the mean values of the relative light units from quadruple determinations as circles with standard deviations. The values for testosterone without addition of the SARM are shown as triangles. On the nonspecific MMTV-promoter (A+B) the SARM tested here behaves as pure antagonist of AR-mediated transactivation, whereas on the neuronal 1f-aromatase promoter (C+D) the partial agonistic/partial antagonistic action comes to the fore.

1. Promoter-reporter gene fusion consisting of 1f-aromatase promoter according to Seq ID #1 and reporter gene.

2. Promoter-reporter gene fusion according to claim 1, characterized in that the reporter gene can be selected from the group comprising a firefly or Renilla luciferase gene and in that the fusion product can be expressed stably or transiently in a cell line.

3. Use of the promoter-reporter gene fusion according to claim 1, characterized in that, in a cell culture system, substances can be identified that have an influence on aromatase regulation in neuronal tissues by binding to the androgen receptor.

4. Use according to claim 3, characterized in that sequences having >90%, preferably >95% homology to the sequence stated under Seq ID # 1 are used in a cell culture system for identifying substances that have an influence on aromatase regulation in the brain by binding to the androgen receptor.

5. Determination of the tissue-selective action of substances that bind to the androgen receptor, comprising the following steps:
a. Determination of the modulation of aromatase regulation using the promoter-reporter gene fusion in a reporter gene assay in cell culture systems for identifying both agonistic and antagonistic activities,
b. Determination of the modulation of another androgen-regulated promoter and
c. Comparison of step a and b, to achieve a complete in vitro characterization of substances with respect to selective AR modulation on the 1f-aromatase promoter or in direct comparison with other AR-regulated promoters.

6. Determination of the tissue-selective action of substances according to claim 5, characterized in that sequences having >90%, preferably >95% homology to the sequence stated under Seq ID # 1 are used in step a).

7. In vitro test system for the identification and characterization of substances that bring about an increase in aromatase expression selectively in the central nervous system and/or are suitable for exerting a tissue-selective influence on the activity of the AR, characterized in that the test system contains a fusion of reporter gene and aromatase promoter according to claim 1.
